(19) United States
(12) Patent Application Publication
(10) Pub. No.: US 2007/0090014 A1
(43) Pub. Date: Apr. 26, 2007

(54) Discreet personal care product kit

(75) Inventors: Katherine Carol Wheeler, Menasha, WI (US); Kristi Jo Bryant, Appleton, WI (US); Herb Flores Velazquez, Neenah, WI (US); Teresa Marie Zander, Bonduel, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/256,780

(22) Filed: Oct. 24, 2005

(51) Int. Cl. A61B 19/02

(52) U.S. Cl. 206/581; 206/438

(57) ABSTRACT

The present invention provides a discreet sanitary kit which allows a person in need of sanitary personal care products to carry the sanitary personal care articles in a manner which will not alert others that the kit contains sanitary articles. Also provided by the present invention is a discreet sanitary kit with a self-indulgence item.

### FIELD OF THE INVENTION

The present invention relates generally to a personal care product kit which provides a person in need of personal care products with a discreet, self-contained kit. The present invention also provides to a person in need of personal care products an item of self indulgence, thereby providing emotional support to the person in need of personal care products.

### BACKGROUND OF THE INVENTION

Absorbent personal care articles are generally known in the art as products of a personal hygiene or health care nature. Such products include, for example, incontinence articles, feminine hygiene products, and the like. These absorbent personal care articles have been generally provided to users in bulk, via containers, such as paper containers, generally in the form of a box or carton, or soft side packages such as polymer film bags. In order for a user to carry these items with them so that they are readily available in a time of need, the user must remove the personal care articles from the packaging and place the articles into a purse, pocket or other container, such as a tongue and groove locking bag or the like. This can often present problems for users of these products. For example, the user must remember to refill the purse or other container so that the personal care items are readily available during a time of need, or the user must anticipate a need for these products and ensure that the products are available by also filling the purse. In addition, the user may be embarrassed as the user attempts to retrieve the personal care product from a purse, pocket or locking bag, since these products may come in plain view for others to see as these products are removed.

Pre-teen and teenage girls experiencing their first few menstrual cycles often do not know what personal care articles they may need just before, during and just after their menstrual cycle. Kits containing the essentials that a menstruating female may need to deal with their menstrual cycles, including items such as tampons, sanitary napkins, or liners, have been suggested by those skilled in the art; however many of these kits, while providing convenience and instructions on how to deal with menstrual cycles, do not provide the pre-teen and teenage girls with a discreet way to carry the essential items needed to deal with their menstrual cycle.

Discretion during a teen or woman's period is always a must, but the discretion must be provided in a convenient manner. Often the discretion is given at the expense of convenience or convenience is provided at the expense of discretion. Many times, particularly in public places, a woman or teenager will carry personal care products such as a sanitary napkin, a tampon, a pantiliner or cleansing products in a large purse and/or will carry the items in her hand to the restroom to make any necessary product changes. When these personal care products are in a large purse, these products tend to fall to the bottom of the purse. This will force the woman or teenager to dig through items in the purse, often causing the user to make the distinguishing sound of rummaging through the purse to locate the needed personal care product. As a result, others in the public restroom will know, by the sounds or action of the user, that the user is in need of the sanitary personal care products, which can often lead to embarrassment for the user. The same is true for products carried in the hand of the user. The user will have a distinctive hand position as she tries to hide the item from the view of others, thereby signaling to others watching that she is in need of a sanitary product. In a similar manner, many users of incontinence products also experience a similar indiscretion.

Previously available personal care product carriers did not have a purse-like appearance and were generally wallets or oblong case type carriers. As a result, a casual observer could easily determine the contents of these wallets or cases by the shape and size of these wallets or cases. Other purse-like containers suggested in the art had side compartments which would allow non-users or casual observers to determine that the purse-like container was not a purse, as may have appeared at first glance. As a result, the art does not provide a discreet carrier for personal care products and other items of personal hygiene and well being.

There is a need in the art to provide a discreet carrying package with all of the essentials needed for teens and women during their menstrual cycle that provides both discretion and convenience. Likewise, there is a need in the art to provide discretion and convenience to users of incontinence products. There is also a need in the art to provide pre-teen and teenagers, who may be experiencing their first few menstrual cycles with an essentials kit, containing items which they may need just before, during or just after their menstrual cycles, as well as provide the pre-teen or teenager with instructions on how to use the product on a level they will be able to understand.

Further, previously provided personal care product kits provided the essentials, i.e., wipes, pain relief, absorbent articles, to deal with menstrual cycles. During menstrual cycles, women and teens often feel sluggish, bloated and unfresh. There are several products on the market which are available that can help a woman or teens feel better about themselves. However, these products are not provided with absorbent personal care articles. There is also a need in the art to provide teens and women a self indulgence type kit, containing items which can make going through their menstrual cycle easier and allow the women or teen to feel fresh and good about themselves during their menstrual cycle.

### SUMMARY OF THE INVENTION

Generally stated, the present invention provides a discreet sanitary kit which allows a person in need of sanitary personal care products to carry the sanitary personal care articles in a manner which will not alert others that the kit contains sanitary articles. The kit has a carrier which has at least a front panel, and a back panel. Each panel having a bottom edge, a first side edge and a second side edge and a top edge, wherein the bottom edge of the front panel is directly or indirectly connected to the bottom edge of the back panel, the first side edge of the front panel is directly or indirectly attached to the first side edge of the back panel, and the second side edge of the front panel is directly or indirectly connected to the second side edge of the back panel. The front panel and the back panel define a compartment capable of holding personal care products, including absorbent personal care articles. Access to the compartment is provided between the top edge of the front panel and top edge of the back panel. The carrier is also provided with a closing mechanism which, when closed, blocks access to the compartment and, when opened, allows access to the compartment. Stored within the compartment is at least two absorbent personal care products, each individually wrapped in a wrapping component. The carrier has an exterior surface and the exterior surface is devoid of any indicia which tend to indicate that the carrier has at least one absorbent personal care article placed within the compartment of the carrier. The carrier is prepared from a material which allows the carrier to have a purse-like appearance and can be prepared from materials having a fabric-like, a leather-like or a hard-case-like appearance.

In another embodiment of the present invention, provided is a discreet sanitary kit. The kit contains a carrier, at least two absorbent articles and at least one self-indulgence item. The carrier has a front panel, a back panel, each panel having a bottom edge, a first side edge and a second side edge and a top edge, wherein the bottom edge of the front panel is directly or indirectly attached to the bottom edge of the back panel, the first side edge of the front panel is directly or indirectly attached to the first side edge of the back panel, and the second side edge of the front panel is directly or indirectly attached to the second side edge of the back panel. In addition, the front panel and the back panel define a compartment capable of holding absorbent personal care products and the self-indulgence item. Access to the compartment is provided between the top edge of the front panel and the back panel. The front and back panels each have an exterior surface, wherein the exterior surface of each of the front and back panels is devoid of any indicia which tends to indicate that the carrier has at least one absorbent personal care article placed within said compartment of the carrier.

### BRIEF DESCRIPTION OF THE DRAWINGS

FIG. 1 shows a representative discreet sanitary kit of the present invention in an opened position.

FIG. 2 shows a representative discreet sanitary kit of the present invention in a closed position.

FIG. 3 shows another representative discreet sanitary kit of the present invention in a closed position.

FIG. 4 shows a perspective view of a self-indulgence/absorbent article kit of the present invention.

FIG. 5A shows a possible configuration for an absorbent personal care article placed in the wrapper component of the present invention.

FIG. 5B is a perspective view of a product component in a partially folded configuration.

FIG. 6 shows a plan view of the construction of a tampon component of the present invention.

FIG. 7 shows a perspective view of an uncompress tampon of the present invention.

FIG. 8 shows a perspective view of a tampon component of the present invention.

FIG. 9 shows a perspective view of a tampon applicator of the present invention.

### DEFINITIONS

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

It should be understood that the term “personal care product” or “personal care article” as used herein refers to any article used to control bodily fluids, and includes “absorbent products,” which refers to any article configured to absorb and retain bodily exudates, including urine, blood and menses, and includes such a product in a packaged and unpackaged configuration. As such, “personal care products” as used herein, includes without limitation, tampons, vaginal suppositories, pantiliners, pads, sanitary napkins, tissues, wipes and similar items to deal with bodily discharges. For example, personal care products include, without limitation, Poise® feminine care products, including pantiliners and pads, and Kotex® feminine care products, including sanitary napkins, tampons and liners, all available from Kimberly-Clark Corporation, Neenah, Wis.

As used herein, the term “connected” is intended to mean that two or more members are directly or indirectly connected, to one another. When two or more members are directly connected to one another, it is meant that the two members are in direct contact with one another, without an intervening member or structure. When two or more members are indirectly connected to one another, it is meant that the two members are not in direct contact with one another, and may have an intervening member or structure between the two or more members connected to one another.

The phrase “devoid of any indicia which tends to indicate that the package has at least one absorbent personal care product” is intended to mean that the presence of words, symbols, pictures and other marks which would suggest the possible presence of an absorbent personal care product within the package are not present on the defined walls and panels. Examples of indicia intended to be excluded include, brand names, words or phrases normally associated with absorbent articles, such as “liners”, “pantiliners”, “tampons”, “sanitary napkins” and the like, or symbols or pictures which depict such absorbent personal care articles. It is noted that this phrase is not intended to exclude information or indicia, which does not imply the presence of an absorbent personal care article, for example, but not limited to, flowers, patterns, or pictures not associated with absorbent personal care articles, and the like.

As used herein, the term “self indulgence item” refers to an item which is not necessary for a female user of absorbent personal care articles to use during the users menstrual cycle, but is an item which helps the self-image of the user. Self indulgence items include, for example, a candy, aromatherapy items, a bath product such as a bath soap, a bath oil, a bath gel, bath petals or bath beads, a candle, inscents, a compact disc containing soothing music, discs containing various scents, such as Febreeze™ Scentstories™, perfumes, body lotions, body sprays, body oils, body creams, body mists, a book, such as a romance novel, a lip balm, lipstick or other make-up items, such as eyeliner, eye shadow, a facial peel, foot scrub, a booklet with words of encouragement, or other similar items that relax and/or soothe a user.

### DETAILED DESCRIPTION OF THE INVENTION

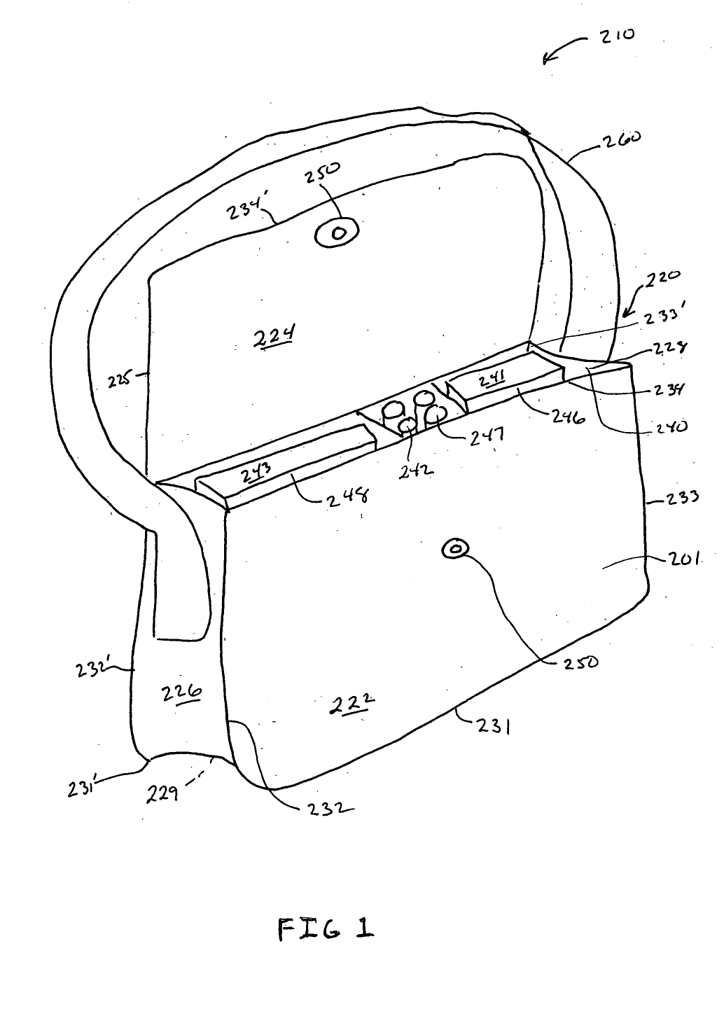

To obtain better understanding of the present invention, attention is directed to the Figures. As shown in FIG. 1-3, a discreet sanitary kit 210 which allows a person in need of sanitary personal care products to carry the sanitary personal care articles in a manner which will not alert others that the kit contains sanitary articles. The kit 210 has a carrier 220 which has at least a front panel 222, and a back panel 224. Each panel having a bottom edge 231, 231′, a first side edge 232, 232′, and a second side edge 233, 233′ and a top edge 234, 234′. The bottom edge 231 of the front panel 222 is directly or indirectly connected to the bottom edge 231′ of the back panel 224, the first side edge 232 of the front panel 222 is directly or indirectly attached to the first side edge 232′ of the back panel 224, and the second side edge 233 of the front panel 222 is directly or indirectly connected to the second side edge 233′ of the back panel 224. The front panel 222 and the back panel 224 define a compartment 240 having sufficient volume to hold personal care products 241, 242, 243, including absorbent personal care articles. Although shown in FIG. 1 as three separate personal care products, the compartment 240 may hold as few as one type of personal care product or many different types of personal care products. A description of the personal care products which may be placed within the compartment are described below in more detail. Access to the compartment is provided between the top edge 234 of the front panel 222 and top edge 234′ of the back panel 224. The carrier of the present invention may have a purse-like appearance.

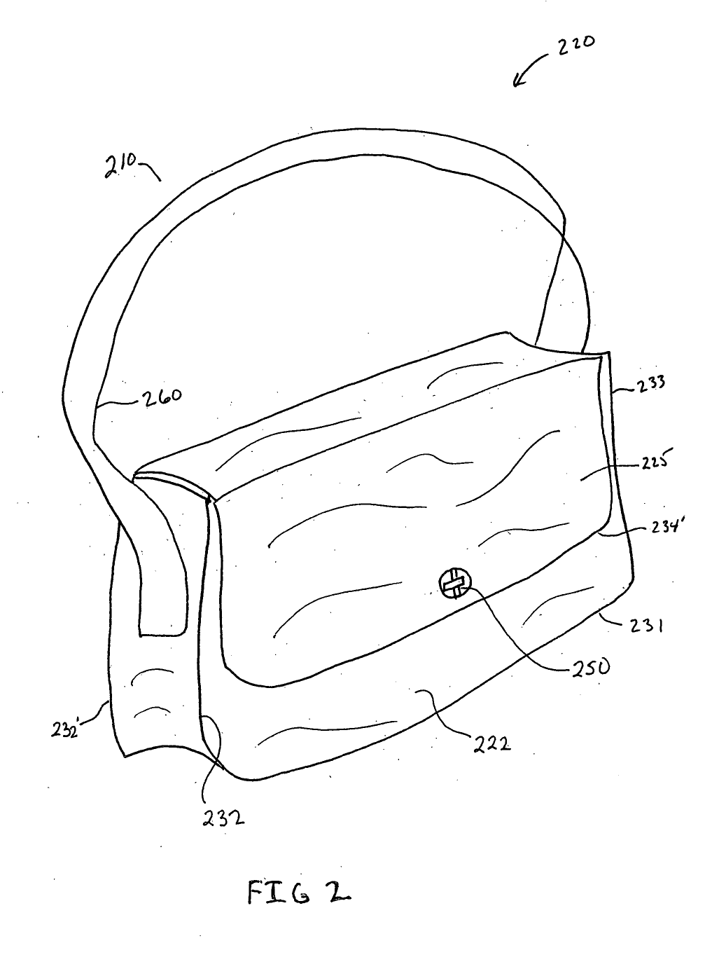

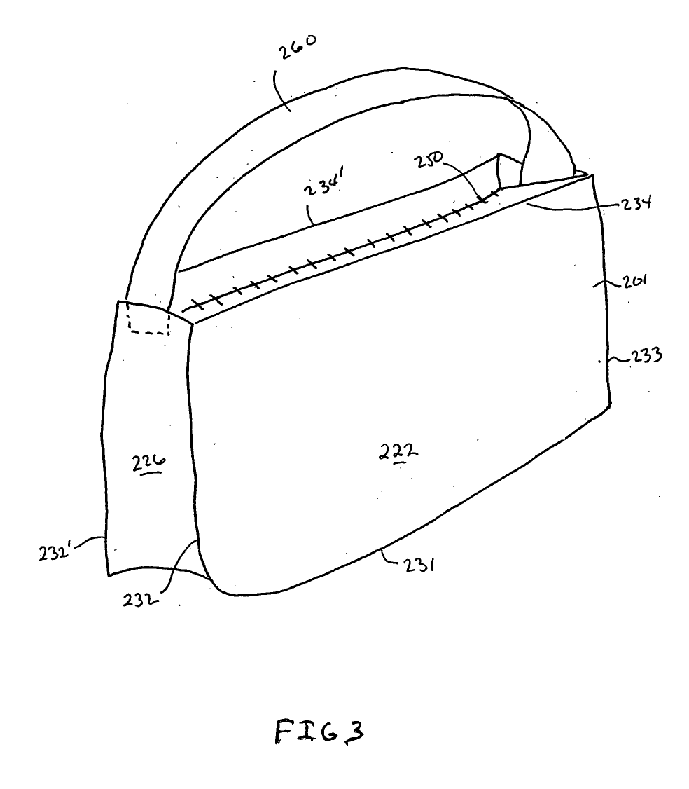

The carrier is also provided with a closing mechanism which, when closed, blocks access to the compartment 240 and, when opened, allows access to the compartment 240. In the case of FIGS. 1 and 2, the closing mechanism is provided by the back panel 224 being longer or taller than the front panel 222. The back panel 224 has sufficient length such that the back panel creates a flap 225. This flap 225 will be able to cover the compartment and the interior surface of the flap 225 and will contact the exterior surface of the front panel 222 in the closed position. Other closing mechanisms may also be used. For example, in FIG. 3, the front panel 222 and the back panel 224 are about the same length. These panels are joined together with a mechanical closing mechanism 251, which is shown in FIG. 3 as a zipper. Other mechanical closing mechanisms may also be used, provided that the closing mechanisms will effectively contain and conceal the personal care products in the carrier 220, without departing from the scope of the present invention.

In the kit of the present invention, stored within the compartment 240, is at least two absorbent personal care products 241. Each of the personal care products may be the same type of personal care products or different types of personal care products. The types of personal care products contained within the compartment 240 of the carrier 220 will be described in more detail below. The personal care products may be individually wrapped in a wrapping component.

The carrier 220 has an exterior surface 201 and the exterior surface 201 is devoid of any indicia which tends to indicate that the carrier 220 has at least one absorbent personal care article placed within the compartment 240 of the carrier 220. For example, the exterior surface is devoid of words, symbols, pictures and other marks which would suggest the possible presence of an absorbent personal care product within the carrier.

In addition to the front and back panels 222, 224, the carrier 220 may also have a first side panel 226 and a second side panel 228. The first side panel 226 joins the first side edge 232 of the front panel 222 to the first side panel 232′ of the back panel 224. Similarly, the second side panel 228 joins the second side edge 233 of the front panel 222 to the second side edge 232′ of the back panel 224. In addition, there may also be a bottom panel 229, which connects the bottom edge 231 of the front side panel 222 to the bottom side edge 231′ of the back side panel 224. It is noted that the side panels 226 and 228 as well as the bottom panel 229 are optional. Other configurations may also be used for the front and back panels. In one configuration, the front side panel 222 and the back side panel 224 are a single continuous piece of material. When a single piece of material is used for the front and back panels, generally the piece of material is continuous from the top edge 234 to the top edge 234′. This will result in a carrier which has only side seams.

The carrier 240 is prepared from a material which allows the carrier to have a purse-like appearance and can be prepared from materials having a fabric-like, a leather-like or a hard-case like appearance. Examples of materials which may be used for the carrier include fibrous webs and other materials which purses are typically prepared from including, leather, leatherette, vinyl, and the like. Hard sided materials, such as hard plastic cases, are made from durable polymeric materials such as polyethylene, polypropylene, acrylonitrile-butadiene-styrene (ABS) copolymers. Other materials may be used in the present invention, as long as the carrier is made to be discreet and does not relay to non-users that the carrier contains personal care products. In addition, two or more different types of materials may be used together to form the carrier to create desired effects, such as an aesthetic appeal or physical properties. For example, one panel may be a film material or leather-like while another may be a fibrous material. An example of a possible aesthetic appeal that may be obtained in the present invention by using two or more pieces of material to form the package is a quilt or patch-work effect, which will further aid in providing discretion for the carrier.

The fibrous webs usable in the present invention as a material which include woven, knitted or nonwoven fabrics. By making the package out of these materials, an additional benefit of an added level of discretion may be provided, since the package will take on a purse-like appearance. From a standpoint of cost, nonwoven webs are desired over the woven or knitted fabrics. Suitable nonwoven fabrics include, for example, airlaid nonwoven webs, spunbond nonwoven webs, meltblown nonwoven webs, bonded-carded-webs, hydroentangled nonwoven webs, spunlace webs and the like. The method of manufacturing each of these materials is known in the art. Laminates of these materials may also be used.

Of these nonwoven webs, the fibrous material web may comprise a nonwoven meltblown web. Meltblown fibers are formed by extruding a molten thermoplastic material through a plurality offine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al. Generally speaking, meltblown fibers may be microfibers that may be continuous or discontinuous, and are generally smaller than 10 microns in diameter, and are generally tacky when deposited onto a collecting surface.

The fibrous material web may comprise a nonwoven spunbond web. Spunbonded fibers are small diameter substantially continuous fibers that are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spun-bonded nonwoven webs is described and illustrated, for example, in U.S. Pat. No. 4,340,563 to Appel, et al.; U.S. Pat. No. 3,692,618 to Dorschner, et al.; U.S. Pat. No. 3,802,817 to Matsuki et al.; U.S. Pat. No. 3,338,992 to Kinney; U.S. Pat. No. 3,341,394 to Kinney; U.S. Pat. No. 3,502,763 to Hartman; U.S. Pat. No. 3,502,538 to Levy; U.S. Pat. No. 3,542,615 to Dobo, et al.; and U.S. Pat. No. 5,382,400 to Pike, et al. Spunbond fibers are generally not tacky when they are deposited onto a collecting surface. Spunbond fibers can sometimes have diameters less than about 40 microns, and are often between about 5 to about 20 microns.

The fibrous material web may also comprise a laminate material such as a spunbond/meltblown/spunbond, or SMS, material. A typical SMS material is described in U.S. Pat. No. 4,041,203 to Brock et al. Other SMS products and processes are described, for example, in U.S. Pat. No. 5,464,688 to Timmons et al.; U.S. Pat. No. 5,169,706 to Collier et al.; and U.S. Pat. No. 4,766,029 to Brock et al. Generally, an SMS material will consist of a meltblown web sandwiched between two exterior spunbond webs. Such SMS laminates have been available commercially for years from Kimberly-Clark Corporation under marks such as Spunguard® and Evolution®. The spunbonded layers on the SMS laminates provide durability and the internal meltblown layer provides porosity and additional cloth-like feel.

In another aspect of the present invention, the carrier 220 material may be a laminate of a film layer and fibrous material layer which have been laminated together by any lamination technique known to those skilled in the art. Suitable lamination means include, but are not limited to, adhesives, ultrasonic bonding and thermomechanical bonding as through the use of heated calendering rolls. Such calendering rolls will often include a patterned roll and a smooth anvil roll, though both rolls may be patterned or smooth and one, both or none of the rolls may be heated. Calandering may also be used to place an aesthetic pattern defined in the laminated wrapper material. Examples of these laminates include, for example, spunbond-film laminates (SF), and other such laminates. In one embodiment, the material for the wrapper component are prepared from a film/spunbond laminate material available from Kimberly-Clark Corp, and known as HBSTL (“highly breathable stretch thermal laminate”), and which is further disclosed in U.S. Patent No. 6,276,032, the entire disclosure of which is hereby incorporated herein by reference. Other materials such as elastic films and elastic nonwoven webs may also be used to create a chamber which is expandable. Each panel may be prepared from different materials and each panel may be prepared from one or more material types. For example, one panel may be a film and the other panel a nonwoven web. The carrier 220 may also be a multi-ply material or a single ply material.

Selection of the material used to prepare the carrier 220 is not that critical to the present invention, provided that the material selected will provide discretion to the user. The material should be sufficiently opaque so that the material is not transparent or translucent that others can see the products being carried within the compartment 240 of the carrier. In addition, the material should be such that the material will not break, tear or exhibit excessive bulging as to show the outline of the personal care product can be seen from the outside of the carrier 220. From a standpoint of cost, durability and aesthetic value, it is desirable that the fibrous web is a nonwoven web. Of the nonwoven webs listed above, from a durability standpoint, it is desirable that the nonwoven web be a spunbond nonwoven web, or a spunbond containing laminate. One particular spunbond nonwoven web laminate is a SMS (spunbond/meltblown/spunbond) laminate.

Whether the carrier 220 is prepared from a unitary piece of material or from two or more pieces of material, it will be necessary to join the material or materials together at one or more seams. Any method known to those skilled in the art of joining two pieces of material together may be used. Exemplary joining methods include bonding, sealing, stitching and the like. Suitable bonding and sealing methods include, for example, but are not limited to adhesive bonding or sealing, bonding or sealing by the application of heat and pressure, ultrasonic bonding or sealing, or any other bonding methods known to those skilled in the art. The particular method used to form the seams is not critical to the present invention. However, when the materials are thermoplastic based materials, such as films and nonwoven webs, from the standpoint of ease and speed of production, it is desirable to use pressure and heat or ultrasonic bonding or sealing methods.

The carrier 220 of the kit of the present invention is not restricted in its size. Generally, the carrier 220 will be appropriately sized depending on factors such as the type of personal care articles placed into the carrier, and the number of personal care articles placed in the carrier 220. Larger personal care products and higher numbers of personal care products intended to be stored and carried in the carrier 220 will result in a large carrier.

The compartment 240 of the carrier 220 may be a single storage area or may be a series of smaller compartments. As shown in FIG. 1, the compartment 240 has three separate storage areas 246, 247, and 248. Each storage area may be shaped and sized to hold a specific type of personal care article. For example, the storage area 247, as shown in FIG. 1, contains tampons, and is therefore narrower than the adjacent storage areas 246 and 247. The number of storage areas with the compartment 240 of the carrier 220, may be adjusted depending on the number of different personal care articles, types of personal care articles and the number of each type of personal care article carried within the carrier 240. For example, a storage area containing sanitary napkins would have to be larger than a storage area containing pantiliners, assuming that there are similar numbers of each article.

The carrier 220 may also optionally have a handle or strap 260 to provide a way for a user to carry the kit 210 in a discreet fashion, much in the same manner as a purse or a handbag. The handle or strap 260 may be of any length as typically used for womens' purses or handbags. The length of the strap or handle 260 may be sufficiently long such that the strap or handle may be placed over the shoulder, resulting in the carrier 220 being carried at or near the hip of the user. Alternatively, the strap or handle 260 may be shorter resulting in a carrier 220 to be carried by the user in her hand, such as a handbag. The handle or strap 260 may be attached to an interior surface of the carrier 220 or may be attached to the exterior surface of the carrier. Typically, the handle or strap will be attached to the exterior surface of the carrier, as is shown in FIGS. 1-3. Any method known to those skilled in the art may be used to attach the handle or strap 260 to the carrier 220. The handle or strap 260 may be directly attached to one of the panels of the carrier, as is shown in FIGS. 1-3. As an alternative to directly attaching the handle or strap 260 to one of the panels of the carrier 220, a loop or other mechanism (not shown) allowing the strap or handle 260 to be looped through or attached may also be used. The loop or other mechanism may be attached directly to one of the panels of the carrier 220 and the strap or handle 260 may be looped through or attached to this other attachment mechanism. As is shown in FIGS. 1 and 2, the strap or handle 260 is directly attached to an exterior surface on some of the panels, in particular, the exterior surface of the side panels 226 and 228. In FIG. 3, the strap or handle 260 is attached to an interior surface of the side panels 226 and 228. The handle or strap 260 is located in a manner such that the handle or strap 260 extends beyond the top edge 234 of the front panel 222. Stated another way, the handle or strap should extend in an upward manner above the opening to the compartment 240 of the carrier 220. Generally, the handle or strap 260 should be positioned on the carrier 220 in such a manner that the strap or handle 260 may be moved away from the opening of the compartment 240, by the user, to facilitate access to the compartment and the personal care article stored within the compartment 240. The actual method or location of the attachment of the handle or strap 260 to the carrier 220 may be done for aesthetic reasons. The strap or handle 260 also serves as a hanging means for the kit when it is placed or used in a restroom stall and the like.

The material used for the strap or handle 260 should have sufficient strength to carry the weight of the carrier with personal care products contained therein. In addition, the strap material should be durable enough to withstand repeated uses over a period of time. In addition, the strap or handle 260 should be coordinated with the carrier, meaning that the strap or handle should look like a part of the carrier 220. As a result, suitable materials usable for the strap or handle include many of the same materials used to prepare the carrier. Ideally, the strap or handle 260 is prepared from the same material as the carrier. From a standpoint of cost, durability and aesthetic value, it is desirable that the handle or strap 260 is prepared from a nonwoven web. Of the nonwoven webs listed above for the carrier, from a durability standpoint, it is desirable that the nonwoven web for the handle or strap 260 be a spunbond nonwoven web, or a spunbond containing laminate. One particular spunbond nonwoven web laminate is a SMS (spunbond/meltblown/spunbond) laminate.

The carrier may also be provided with a locking mechanism 250 that will prevent the carrier from opening unexpectedly. Examples of possible locking mechanisms include, for example, closing mechanism comprises a zipper, a buckle, a button, a snap, a hook and loop fastener (for example VELCRO® type fasteners), a string fastener or a latch. Other similar locking mechanisms conventionally used on purses and hand bags may also be used in the present invention. It is noted that the locking mechanism 250 may also serve as a closing mechanism described above.

Although the carrier is devoid of any indicia on the exterior surface, which tends to indicate the presence of absorbent personal care articles, does not mean that the carrier is devoid of all graphics. Pictures and designs unrelated to personal care articles may be present on the exterior surface of the package. In addition, the interior surface of the flap of the carrier, if present, may be imparted with information on how,to use the personal care products placed within the package or other information, such as words of inspiration to the user of the personal care products. Another way of conveying needed information to the user includes placing an insert in the package with instructions on how to use the products.

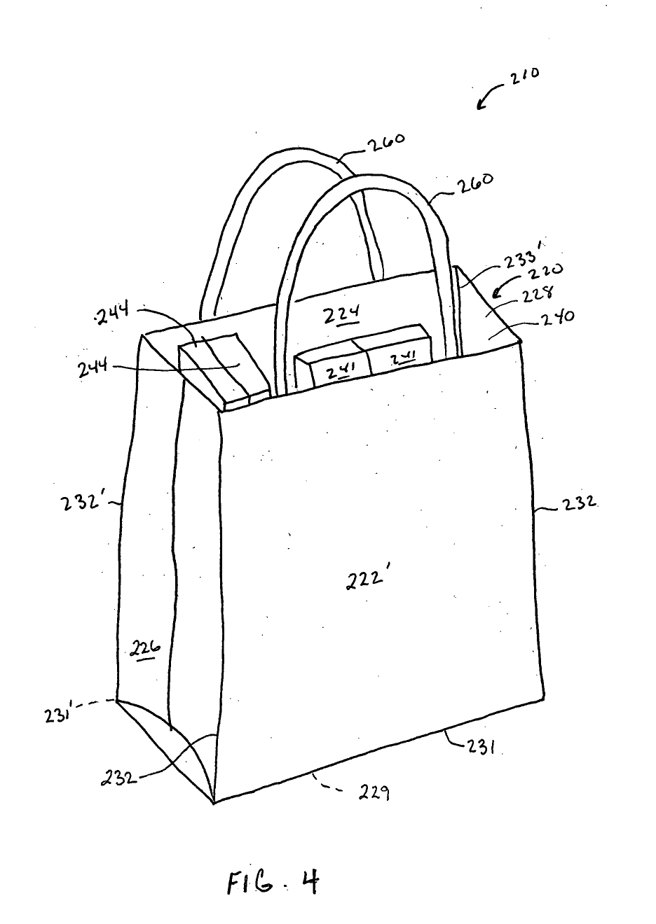

An alternative embodiment of the present invention is shown in FIG. 4, which shows a bag-like appearance for the carrier. In FIG. 4, the kit 210 has a carrier 220 which has at least a front panel 222, and a back panel 224. Each panel having a bottom edge 231, 231′, a first side edge 232, 232′, and a second side edge 233, 233′ and a top edge 234, 234′. The bottom edge 231 of the front panel 222 is directly or indirectly connected to the bottom edge 231′ of the back panel 224, the first side edge 232 of the front panel 222 is directly or indirectly attached to the first side edge 232′ of the back panel 224, and the second side edge 233 of the front panel 222 is directly or indirectly connected to the second side edge 233′ of the back panel 224. The front panel 222 and the back panel 224 define a compartment 240 having sufficient volume to hold personal care products 241, 242, 243, including absorbent personal care articles, and at least one self-indulgence product 244. In addition to the front and back panels 222, 224, the carrier 220 may also have a first side panel 226 and a second side panel 228. The first side panel 226 joins the first side edge 232 of the front panel 222 to the first side panel 232′ of the back panel 224. Similarly, the second side panel 228 joins the second side edge 233 of the front panel 222 to the second side edge 232′ of the back panel 224. In addition, there may also be a bottom panel 229, which connects the bottom edge 231 of the front side panel 222 to the bottom side edge 231′ of the back side panel 224. It is noted that the side panels 226 and 228 as well as the bottom panel 229 are optional. Other configurations may also be used for the front and back panels. In one configuration, the front side panel 222 and the back side panel 224 are a single continuous piece of material. When a single piece of material is used for the front and back panels, generally the piece of material is continuous from the top edge 234 to the top edge 234′. This will result in a carrier which has only side seams. In the configuration of the kit, the carrier maybe prepared from a film material, a cellulose based material, such as paper, cardboard and the like. The carrier may be in the form of a bag or a box. The carrier 220 may also optionally have a handle or strap 260 to provide a way for a user to carry the kit 210 in a discreet fashion, much in the same manner as a bag.

The personal care articles placed in the carrier may be absorbent personal care articles. These absorbent personal care articles include, for example, feminine napkins (sanitary napkins), pantiliners, tampons, interlabial pads, other feminine care products, and adult care products.

Absorbent articles such as, for example, feminine care and incontinent absorbent products, generally include a liquid pervious topsheet, a substantially liquid impervious backsheet, and an absorbent core positioned and held between the topsheet and the backsheet. The topsheet is generally operatively permeable to the liquids that are intended to be held or stored by the absorbent article, and the backsheet may be substantially impermeable or otherwise operatively impermeable to the liquids intended to be held or stored. Disposable absorbent articles may also include other optional components or layers, such as liquid wicking layers, liquid distribution layers, barrier layers, and the like, as well as combinations thereof, which may improve the fluid handling and storage properties of the disposable absorbent article. Generally, disposable absorbent articles and the components thereof, provide a body-facing surface and a garment-facing surface. As an alternative, the substantially liquid impervious backsheet may be replaced with a liquid pervious backsheet, when the absorbent personal care product is used in conjunction with another liquid impervious layer or article, such as, for example liquid impervious pants. Absorbent articles are exemplified in numerous U.S. Patents and patent applications, including, U.S. patent application Ser. No. 10/379,942, entitled “Perimeter Embossing in an Absorbent Article”, filed Mar. 4, 2003; U.S. patent application Ser. No. 10/392,116, entitled “Multilayer Absorbent Article”, filed Mar. 19, 2003; and U.S. patent application Ser. No. 10/753,974, entitled “Low Profile Absorbent Pantiliner” filed Jan. 7, 2004, all of which are hereby incorporated by reference in their entirety.

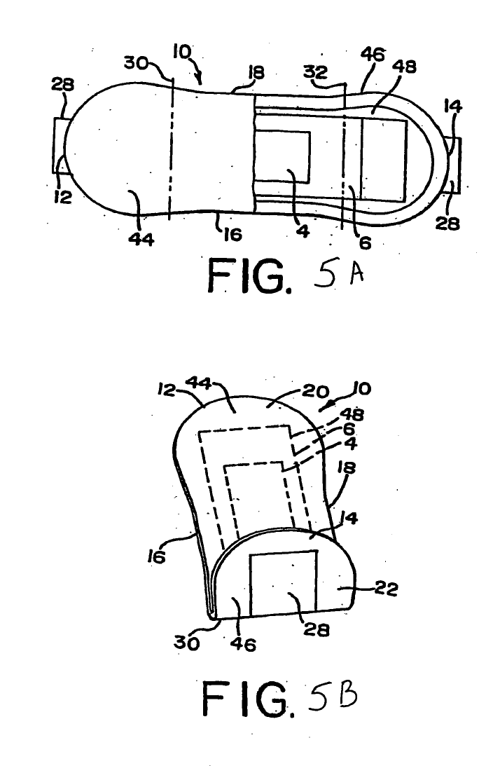

To obtain a better understanding of the absorbent articles which may be present in the carrier 220 of the present invention, attention is directed to FIGS. 5A and 5B. In FIGS. 5A and 5B, an exemplary absorbent product 10, is shown as including an outer cover 46 (otherwise referred to as a baffle or backsheet), an absorbent core 48, an optional tissue layer 6, an optional distribution layer 4 and a body-side liner 44 (also referred to as the top sheet). The absorbent product 10 also has a first side 16 and a second side 18. The first and second sides 16, 18, respectively, are the longitudinal sides of the elongated absorbent product. The sides can be contoured, for example, in a concave shape, or they can be linear. The sides can further include flaps (not shown) that extend laterally outward. Flaps are known in the art and are shown in, for example, U.S. Pat. No. 6,387,084 issued to VanGompel et al. or U.S. Pat. No. 4,589,876, issued to Van Tillburg, which are hereby incorporated by reference for its discussion of the flaps and flap attachment means and in its entirety. In one embodiment (not shown), one or more elastic elements are disposed along the sides to form a gasket with the body of the user. Elastic sides are known in the art, as is shown in U.S. Pat. 6,315,765 issued to Datta et al., which is hereby incorporated by reference for its discussion of the elastic sides and in its entirety. In one embodiment, the elastic elements are disposed between the liner and the outer cover.

The absorbent product 10 has a first body-side surface 20 and a second garment side surface 22. Applied to at least a portion of the second garment side surface 22 is a garment attachment adhesive. In various embodiments, the garment attachment adhesive is configured as a single band of adhesive or as two or more spaced apart strips. Alternatively, the garment attachment adhesive includes a swirl pattern of adhesive which encompasses a major portion of the second garment surface 22 of the absorbent article 10.

A release strip 28, also known as a releasable peel strip, is removably secured to the garment attachment adhesive and serves to prevent premature contamination of the adhesive before the absorbent article 10 is secured to, for example, the crotch portion of an undergarment. In various embodiments, the garment attachment adhesive is designed to be secured to the inner crotch portion of an undergarment so as to keep the absorbent product in register with the body of the user. The release strip 28 may extend beyond one or both of the ends 12, 14 of the outer cover, as shown in FIGS. 5A and 5B.

The body-side liner or topsheet 44, which is preferably liquid permeable, may be formed from one or more materials. The body-side liner or topsheet 44 must be able to manage different body excretions depending on the type of product. In feminine care products, often the liner or body contacting layer must be able to handle menses and urine. In the present invention, the body-side liner or topsheet 44 may include a layer constructed of any operative material, and may be a composite material. For example, the liner or body contacting layer can include a woven fabric, a nonwoven fabric, a polymeric film, a film-nonwoven fabric laminate or the like, as well as combinations thereof. Examples of a nonwoven fabric useable in the body-side liner or topsheet 44 include, for example, an airlaid nonwoven web, spunbond nonwoven web, meltblown nonwoven web, a bonded-carded-web, hydroentangled nonwoven webs, spunlace webs or the like, as well as combinations thereof. Other examples of suitable materials for constructing the body-side liner or topsheet 44 can include rayon, bonded carded webs of polyester, polypropylene, polyethylene, nylon, or other heat-bondable fibers finely perforated film webs, net-like materials, and the like, as well as combinations thereof. These webs can be prepared from polymeric materials such as, for example, polyolefins, such as polypropylene and polyethylene and copolymers thereof, polyesters in general including aliphatic esters such as polylactic acid, nylon or any other heat bondable materials.

Other examples of suitable materials for the body-side liner or topsheet 44 are composite materials of a polymeric film and a nonwoven fabric material. The composite materials are typically in the form of integral sheets generally formed by the extrusion of a polymer onto a nonwoven web, such as a spunbond material. In one arrangement, the liner or body contacting layer 44 can be configured to be operatively liquid-permeable with regard to the liquids that the article is intended to absorb or otherwise handle. The operative liquid-permeability may, for example, be provided by a plurality of pores, perforations, apertures or other openings, as well as combinations thereof, which are present or formed in the liner or body contacting layer. The apertures or other openings can help increase the rate at which bodily liquids can move through the thickness of the liner or body contacting layer and penetrate into the other components of the article (e.g. into the absorbent core 48). The selected arrangement of liquid-permeability is desirably present at least on an operative portion of the body-side liner or topsheet 44 that is appointed for placement on the body-side of the article. The body-side liner or topsheet 44 can provide comfort and conformability, and can function to direct bodily exudates away from the body and toward the absorbent core 48. The body-side liner or topsheet 44 can be configured to retain little or no liquid in its structure, and can be configured to provide a relatively comfortable and non-irritating surface next to the body-tissues of a female wearer.

The baffle or backsheet 46 may include a layer constructed of any operative material, and may or may not have a selected level of liquid-permeability or liquid-impermeability, as desired. In a particular configuration, the baffle or backsheet 46 may be configured to provide an operatively liquid-impermeable baffle structure. The baffle or backsheet 46 may, for example, include a polymeric film, a woven fabric, a nonwoven fabric or the like, as well as combinations or composites thereof. For example, the baffle may include a polymeric film laminated to a woven or nonwoven fabric. In a particular feature, the polymeric film can be composed of polyethylene, polypropylene, polyester or the like, as well as combinations thereof. Additionally, the polymeric film may be micro-embossed, have a printed design, have a printed message to the consumer, and/or may be at least partially colored. Suitably, the baffle or backsheet 46 can operatively permit a sufficient passage of air and moisture vapor out of the article, particularly out of an absorbent (e.g. storage or absorbent core 48) while blocking the passage of bodily liquids. An example of a suitable baffle material can include a breathable, microporous film, such as those described in, for example, U.S. Pat. No. 6,045,900 to McCormack et al.

Bicomponent films or other multi-component films can also be used, as well as woven and/or nonwoven fabrics which have been treated to render them operatively liquid-impermeable. Another suitable baffle material can include a closed cell polyolefin foam. Examples of closed cell foams include a closed cell polyethylene foam.

The liquid permeable body-side liner 44 and the liquid-impermeable baffle 46 may be peripherally sealed together to enclose the absorbent core 48 to form the absorbent article 10. Alternatively, the body-side liner or topsheet 44 can be wrapped around both the absorbent 48 and the baffle or backsheet 46 to form a wrapped pad. The body-side liner 44 and baffle 46, and other components of the absorbent product, can be joined, for example, with adhesive bonds, sonic bonds, thermal bonds, pinning, stitching or any other attachment techniques known in the art, as well as combinations thereof.

The absorbent core 48 is designed to absorb bodily exudates, including menstrual fluid, blood, urine, and other body fluids. The absorbent core 48 may contain one or more layers of absorbent material. The layers can contain similar materials or different materials. Suitable materials for the absorbent core 48 include, for example, cellulose, wood pulp fluff, rayon, cotton, and meltblown polymers such as polyester, polypropylene or coform. Coform is a meltblown air-formed combination of meltblown polymers, such as polypropylene, and absorbent staple fibers, such as cellulose. A preferred material is wood pulp fluff, for it is low in cost, relatively easy to form, and has good absorbency.

The absorbent core 48 can also be formed from a composite comprised of a hydrophilic material which may be formed from various natural or synthetic fibers, wood pulp fibers, regenerated cellulose or cotton fibers, or a blend of pulp and other fibers. In one embodiment of the present invention, the absorbent may be an airlaid material.

In one embodiment, the absorbent core 48 also includes a superabsorbent material, in addition to or in place of the hydrophilic material, which increases the ability of the absorbent core to absorb a large amount of fluid in relation to its own weight. Generally stated, the superabsorbent material can be a water-swellable, generally water-insoluble, hydrogel-forming polymeric absorbent material, which is capable of absorbing at least about 15, suitably about 30, and possibly about 60 times or more its weight in physiological saline (e.g. saline with 0.9 wt % NaCl). The superabsorbent materials can be inserted as particles or in sheet form. The superabsorbent material may be biodegradable or bipolar. The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers may be lightly crosslinked to render the material substantially water insoluble. Crosslinking may, for example, be by irradiation or covalent, ionic, Van der Waals, or hydrogen bonding. Hydroxyfunctional polymers have been found to be good superabsorbents for sanitary napkins. Such superabsorbents are commercially available from Dow Chemical; Hoechst-Celanese, and Stockhausen, Incorporated, among others, and are a partially neutralized salt of cross-linked copolymer of polyacrylic acid and polyvinyl alcohol having an absorbency under load value above 25 grams of absorbed liquid per gram of absorbent material (g/g). Other types of superabsorbent materials known to those skilled in the art can also be used.

Additional layers or substrates, including for example, the liquid acquisition and distribution layer 5, also referred to as a surge or transfer layer, and a tissue layer 6 are also incorporated into the absorbent product, for example, between the body-side liner or topsheet 44 and the absorbent core 48. The distribution layer 5 may be shorter than the absorbent core 48 or have the same length as the absorbent core 48. The distribution layer serves to temporarily hold an insulting fluid to allow the absorbent core sufficient time to absorb the fluid, especially when a superabsorbent material is present. In one embodiment, the absorbent core, transfer layer and other components, such as tissue layers, are free floating (unattached) between the outer cover and the liner, which are secured along only the peripheral edges thereof. Alternatively, the absorbent core, transfer layer and other components are attached to one or both of the outercover and liner and/or to each other.

The absorbent article may be folded along a pair of fold lines 30, 32 to form a tri-fold configuration, prior to insertion into the wrapper component. In other embodiments, the absorbent article can be bi-folded, flat or rolled. In whichever configuration the absorbent article is in, the absorbent article is placed into a wrapper component of the present invention. Alternatively, a plurality (meaning two or more) of article components can be disposed in a single wrapper component and a plurality of wrapper components, with absorbent article contained therein, and can be placed into the carrier of the present invention.

Other absorbent articles which may be placed in the compartment of the carrier included catamenial tampons, which may included applicator tampons or digital tampons, also known as non-applicator tampons.

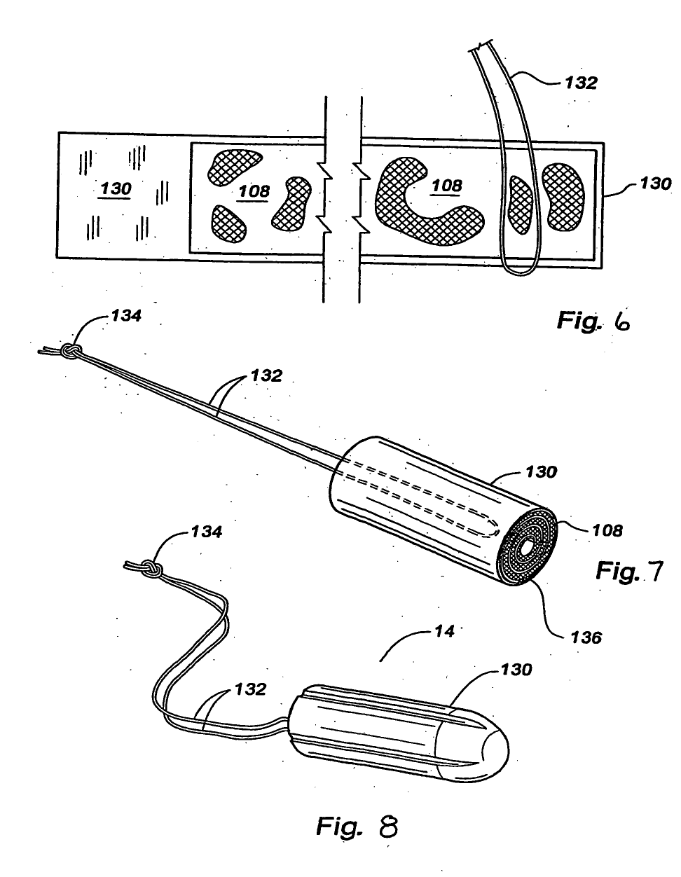

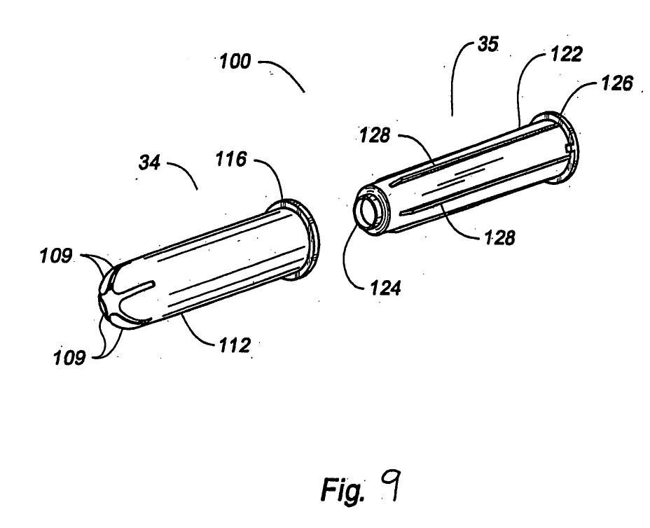

Referring now to FIGS. 6-8, the construction of a catamenial tampon 14 component of the present invention is shown. The tampon 14 includes an absorbent 108 which has been compressed into a generally cylindrical shape and a liquid-permeable cover 130 which surrounds or encloses at least a portion of the absorbent 108. The tampon 14 can be constructed by positioning the absorbent 108 on the cover 130, as indicated in FIG. 6 and then rolling the two layers into a generally cylindrical shape. This uncompressed cylindrical shape, shown in FIG. 7, is known as a “softwind.” The softwind is then compressed into a tampon “pledget” 14 as is shown in FIG. 8. It should be noted that it is also possible first to roll the absorbent 108 into a generally cylindrical shape and then wrap the liquid-permeable cover 130 around it. It also is possible to roll up and compress the absorbent 108 before wrapping it in the cover 130.

The tampon “pledget” as shown as the catamenial tampon 14 in FIG. 8 is an internal sanitary protection device used for absorbing menses, blood, and other body fluids. The tampon 14 is normally used during a woman's menstrual period to prevent leakage of menstrual fluid. The tampon 14 includes an absorbent material, such as cotton, rayon, cellulose wadding, synthetic sponge, cellulose fluff, synthetic fibers, or combinations thereof, which can be compressed into a generally cylindrical shape. The cylindrically shaped absorbent material is usually surrounded by a liquid-permeable cover. Catamenial tampons 14 are available in a variety of sizes and shapes so as to accommodate different size vaginal cavities. The tampons 14 are sufficiently compressed to retain their sizes and shapes under normal atmospheric conditions but are designed to expand readily when wetted by body fluid. For example, after a tampon 14 is inserted into a woman's vaginal tract and is contacted by body fluid, the compressed tampon 14 attempts to re-expand approximately to its original uncompressed size. This structural change allows the tampon to fill the vaginal cavity and absorb body fluid thereby preventing leakage.

Referring to FIG. 8, the tampon 14 further includes a withdrawal string 132 which is secured to either the absorbent 108, the cover 130, or to both and provides a safe and reliable means by which the tampon 14 can be withdrawn from a woman's vagina after it has absorbed a certain amount of menstrual fluid. The withdrawal string 132 can be assembled with the softwind, as depicted in FIG. 7, or it can be attached to the compressed pledget, depending upon one's preference. In either case, the free end of the withdrawal string 132 is tied in a knot 134 to assure that it will not be separated from the pledget.

The absorbent 108 can be formed from absorbent fibers which are first assembled into an absorbent ribbon or sheet. Alternatively, the absorbent 108 can be formed from absorbent fibers which are assembled and compressed into a generally cylindrical configuration. The absorbent 108 is preferably formed from cellulosic fibers, such as cotton and rayon. The absorbent can be 100% cotton, 100% rayon, or a blend of both cotton and rayon. A ratio of from about 15% cotton and about 85% rayon works well. The particular blend of fibers can vary depending on one's preference.

The cotton fibers should have a staple length of between about 5 mm to about 20 mm. The fibers can be bleached if desired. Bleaching will make the fibers whiter in appearance. The cotton should generally have a fiber size of between about 150 to about 280 microns. The rayon fibers should have a staple length of between about 20 mm to about 35 mm. The fibers can also be bleached if desired. The rayon fibers should have a denier of between about 25 to about 28. Denier is a unit of fineness of yarn based on a standard of 50 milligrams per 450 meters of yarn.

The absorbent 108, when formed from an absorbent ribbon, is constructed from a blend of rayon and cotton fibers in a process known to those skilled in the art as “carding.” Depending upon the desired absorbency one desires in the finished tampon, the basis weight of the absorbent ribbon can vary. The U.S. Food and Drug Administration (FDA) has set absorbency standards for “regular,”. “super,” and “super-plus” size tampons. In order to meet these standards for the three above-identified sizes, the absorbent ribbons are targeted to have basis weights of about 127 grams per square meter (g/m²), 170 g/m², and 230 g/m², respectively. Typically, the carding process is controlled to produce an absorbent ribbon with a width of about 50 mm for a “regular” size tampon and a width of about 55 mm for both the “super” and “super-plus” size tampons. During the assembly process, one end of the rectangular absorbent ribbon having a length of about 300 mm is positioned over a portion of the cover 130. The two materials then are rolled up into a generally cylindrical shape. This rolling action will cause the absorbent 108 to be located on the interior and be surrounded by the cover 130. The cover 130 will surround the exterior surface of the softwind and can overlap upon itself, if desired. The cover 130 can be bonded, as indicated by numeral 136, to itself and/or to the absorbent ribbon 108 using heat, pressure, or a combination of heat and pressure. The softwind then is compressed into a finished tampon pledget 14. Preferably, the bonding 136 will occur during the compression step.

The cover 130 can be formed from woven or non-woven materials having a porous substrate. Woven materials include textile fabrics and non-woven materials include spunbond and bonded carded webs.

The withdrawal string 132 can be constructed from various types of threads or ribbons. A thread made from 100 percent cotton fibers works well. The withdrawal string 132 riormally has a length extending beyond one end of the tampon 14 from about 2 inches to about 8 inches (about 50.8 mm to about 203.2 mm), preferably from about 4 inches to about 6 inches (about 102 mm to about 152.4 mm), and most preferably, about 5 inches (127 mm). The withdrawal string 132 can be dyed and treated with an anti-wicking agent, such as wax, before being secured to the softwind or pledget to prevent it from wicking menstrual fluid. A dry, clean withdrawal string 132 is preferred by the user when she goes to remove the tampon 14.

Referring now to FIG. 9, the tampon applicator components of the present invention are constructed to provide a pair of elongated, concentric, telescoping tubes so that the outer tube can carry the tampon's absorbent material, the tampon's “pledget,” while the inner tube serves as a plunger for dispensing the pledget. The tampon applicator components for use in the packaging of the present invention are formed from either plastic or paper. Plastic tampon applicators are preferred by many women because of a molded-in grip ring and a petal-shaped forward end which facilitates insertion of the applicator article while retaining and protecting the tampon while in the outer tube. Plastic tampon applicators typically are formed from polyethylene using injection molding.

Other absorbent articles may include disposable absorbent undergarments or panties, such as those described in U.S. Pat. No. 6,367,089 to Van Gompel et al., which is hereby incorporated by reference.

Other non-absorbent personal care articles, such as wipes, deodorants, pain relievers such as aspirin, acetaminophen, ibuprofen and the like, or unrelated items, such as a self indulgence item, for example, a candy, aromatherapy items, a bath product such as a bath soap, a bath oil, a bath gel, bath petals or bath beads, a candle, inscents, a compact disc containing soothing music, discs containing various scents, perfumes, body lotions, body sprays, body oils, body creams, body mists, a book, such as a romance novel, a lip balm, lipstick or other make-up items as well as other similar items may also be contained in the carrier including, for example, acne relief or acne cover-up. Other items which could be placed in the carrier include a durable pair of replacement panties, so that the pair being worn by the user could be replaced if they were to become soiled due to an unexpected menstrual or incontinence event.

The absorbent articles of the present invention may be placed into the storage compartment of the carrier without being further wrapped in an additional component. In the alternative, the absorbent articles may be wrapped in a wrapper component. Generally, it is desirable to use a wrapper around the absorbent personal care articles since the additional wrapper will help protect and keep sterile the absorbent personal care articles. The wrapper component may have a number of different configurations without departing from the scope of the present invention. These wrapping configurations are well known in the art and any of these known configurations may be used.

The carrier of the present invention may optionally be provided with a fragrance. Many fragrances have colors associated with the fragrance. For example, the color lavender or purple is often associated with a lavender scent; the color yellow is often associated with a chamomile scent, a daffodil and the like; the color red is often associated with a rose scent and the like; and the color green is often associated with the scents of aloe, wintergreen and the like. Other color and scent combinations may be utilized in the present invention.

At the same time, the first color of the personal care product component, such as the absorbent product, can be coordinated with the first color of one or both of the packaging components. In one embodiment, the second color of the personal care product component, such as the absorbent product, is also coordinated with the second color of one or both of the packaging components. The coordination of the colors is most desirable at distances of less than two feet, such that it is visible to the user of the product. At the same time, the coordination can provide a disguising aspect that is effective for an observer who is greater than 2 to 3 feet away from the product. In addition, any designs on the exterior surface of the carrier 220 may also be on the wrapper component for the personal care products. As a result of coordinating the wrapper with the carrier 220, further discretion is provided to the user since the personal care products will tend to blend in visually with the carrier.

In a typical kit of the present invention, there will be 2-8 absorbent products such as sanitary napkins and/or tampons, from 2-20 wipes and one or more other personal care products described above. The kit can be provided to the users as a single kit, with the items already present in the kit, or the carrier can be sold separately from the personal care items, where the personal care items may be sold individually or sold as a group to the user. If the personal care articles are grouped together and sold as group to the user, such as a refill type item, the carrier could be used multiple times. Alternatively, the carrier could be made to be disposable and sold as only an entire kit, i.e. the carrier and articles contained in the carrier.

The kit of the present invention provides discretion to the user of the kit. The discretion provided by the kit 210 of the present invention allows users to discreetly carry personal care products including absorbent personal care products without the stigma, embarrassment and anxiety often associated with carrying these personal care products in public places, such as restrooms and the like.

Those skilled in the art will recognize that the present invention is capable of many modifications and variations without departing from the scope thereof. Accordingly, the detailed description and examples set forth above are meant to be illustrative only and are not intended to limit, in any manner, the scope of the invention as set forth in the appended claims.

1. A discreet sanitary kit comprising
a carrier comprising a front panel, a back panel, each panel having a bottom edge, a first side edge and a second side edge and a top edge, wherein the bottom edge of the front panel is directly or indirectly attached to the bottom edge of the back panel, the first side edge of the front panel is directly or indirectly attached to the first side edge of the back panel, and the second side edge of the front panel is directly or indirectly attached to the second side edge of the back panel, the front panel and the back panel define a compartment capable of holding absorbent personal care products, wherein access to the compartment is provided between the top edge of the front panel and the back panel and a closing mechanism which, when closed, blocks access to the compartment and when opened allows access to the compartment;
at least two absorbent personal care products, each individually wrapped in a wrapping component; said at least two absorbent personal care products being contained within the compartment of the carrier;
wherein the front and back panels each have an exterior surface, wherein the exterior surface of each of the front and back panels is devoid of any indicia which tends to indicate that the carrier has at least one absorbent personal care article placed within said compartment of the carrier and the carrier is prepared from a material having a fabric-like, a leather-like or a hard-case like appearance.

2. The discreet sanitary kit of claim 1, wherein the carrier is prepared from a material having a fabric-like appearance comprising a material selected from a nonwoven web, a woven web, or a knitted material.

3. The discreet sanitary kit of claim 1, wherein further comprising a latching mechanism selected from a zipper, a button, a snap, a latch or hook and loop or string fasteners.

4. The discreet sanitary kit of claim 1, wherein the closing mechanism of the carrier comprises a flap, said flap is an extension of the back panel or is a separate pane which is directly or indirectly connected to the top edge of the back panel, said flap comprises a free end, said free end extends from the back panel and has sufficient length to fold over the top edge of the front panel and contact the outer surface of the front panel.

5. The discreet sanitary kit of claim 4, wherein the flap is an extension of the back panel.

6. The discreet sanitary kit of claim 1, further comprising at least one item selected from wet wipes, dry wipes, pain relief, deodorant spray, acne relief or acne cover-up.

7. The discreet sanitary kit of claim 1, wherein the disposable absorbent article comprises a pantiliner, a sanitary napkin, a disposable undergarment, a incontinence pad or a mixture thereof.

8. The discreet sanitary kit of claim 1, wherein the carrier further comprises a first side panel, a second side panel and a bottom panel, wherein the first side panel joins the first side edge of the front panel to the first side edge of the back panel, the second side panel joins the second side edge of the front panel to the second side edge of the back panel, and the bottom panel joins the bottom edge of the front panel to the bottom edge of the back panel.

9. The discreet sanitary kit of claim 1, wherein the carrier further comprising a handle or strap which allows a user to grasp or carry the carrier.

10. The discreet sanitary kit of claim 1, further comprising at least one self-indulgence item.

11. The discreet sanitary kit of claim 10, wherein the self indulgence item is selected from the group consisting of a candy, an aromatherapy item, a bath product, a perfume, a body lotion, a body spray, a body oil, a body cream, a body mist, a lip balm, lipstick, a make-up item.

12. A discreet sanitary kit comprising a carrier, at least two absorbent articles and at least one self-indulgence item, wherein the carrier comprises a front panel, a back panel, each panel having a bottom edge, a first side edge and a second side edge and a top edge, wherein the bottom edge of the front panel is directly or indirectly attached to the bottom edge of the back panel, the first side edge of the front panel is directly or indirectly attached to the first side edge of the back panel, and the second side edge of the front panel is directly or indirectly attached to the second side edge of the back panel, the front panel and the back panel define a compartment capable of holding absorbent personal care products and the self-indulgence item, wherein access to the compartment is provided between the top edge of the front panel and the back panel, wherein the front and back panels each have an exterior surface, wherein the exterior surface of each of the front and back panels is devoid of any indicia which tends to indicate that the carrier has at least one absorbent personal care article placed within said compartment of the carrier.

13. The discreet sanitary kit of claim 12, wherein the carrier further comprises a first side panel, a second side panel and a bottom panel, wherein the first side panel joins the first side edge of the front panel to the first side edge of the back panel, the second side panel joins the second side edge of the front panel to the second side edge of the back panel, and the bottom panel joins the bottom edge of the front panel to the bottom edge of the back panel.

14. The discreet sanitary kit of claim 13, wherein the carrier is prepared from a material comprising a cellulose-based material.

15. The discreet sanitary kit of claim 14, wherein the carrier comprises a box or a bag.

16. The discreet sanitary kit of claim 12, wherein the self indulgence item is selected from the group consisting of a candy, an aromatherapy item, a bath product, a perfume, a body lotion, a body spray, a body oil, a body cream, a body mist, a lip balm, lipstick, a make-up item.

17. The discreet sanitary kit of claim 12, wherein the self indulgence item is selected from the group consisting of a candy, an aromatherapy item, a bath product, a perfume, a body lotion, a body spray, a body oil, a body cream, a body mist, a lip balm, lipstick a make-up item.

18. The discreet sanitary kit of claim 12, wherein the carrier further comprises a handle or strap which allows a user to grasp or carry -the carrier.

19. The discreet sanitary kit of claim 12, wherein the carrier further comprises a closing device.

20. The discreet sanitary kit of claim 12, wherein the carrier further comprises a first side panel, a second side panel and a bottom panel, wherein the first side panel joins the first side edge of the front panel to the first side edge of the back panel, the second side panel joins the second side edge of the front panel to the second side edge of the back panel, and the bottom panel joins the bottom edge of the front panel to the bottom edge of the back panel, the carrier further comprises a closing device, a handle or strap which allows a user to grasp or carry the carrier and the self indulgence item is selected from the group consisting of a candy, an aromatherapy item, a bath product, a perfume, a body lotion, a body spray, a body oil, a body cream, a body mist, a lip balm, lipstick, and a make-up item.

21. The discreet sanitary kit of claim 12, wherein the at least one absorbent personal care product comprises a sanitary napkin or a pantiliner.
